(19) United States
(12) Patent Application Publication
(10) Pub. No.: US 2016/0067425 A1
(43) Pub. Date: Mar. 10, 2016

(54) DEVICES AND METHODS FOR TREATING TISSUES AND TISSUE CONDITIONS

(71) Applicant: Michael S. Kolodney, Roanoke, VA (US)

(72) Inventor: Michael S. Kolodney, Roanoke, VA (US)

(21) Appl. No.: 14/813,279

(22) Filed: Jul. 30, 2015

Related U.S. Application Data
(60) Provisional application No. 62/031,722, filed on Jul. 31, 2014.

(51) Int. Cl. A61M 5/42; A61B 17/28

(52) U.S. Cl. CPC A61M 5/425; A61B 17/28; A61B 2017/2808

(57) ABSTRACT

Certain embodiments described herein are directed to methods and system that can be used to pressure isolate tissue regions including soft and hard tissues. In some instances, a systemically toxic concentration of an agent can be introduced into a pressure isolated region to treat a disorder of the region. In certain configurations, a system including a pressure member may be used to pressure isolate the region.

### PRIORITY APPLICATION

This application is related to, and claims the benefit of, U.S. Provisional Application No. 62/031,722 filed on Jul. 31, 2014, the entire disclosure of which is hereby incorporated herein by reference for all purposes.

### TECHNOLOGICAL FIELD

This application is related to devices and methods for treating tissues and tissue conditions such as, for example, skin conditions. In certain configurations, a device configured to provide pressure isolation of the tissue region is described. In some instances, the device can also be configured to introduce a therapeutic agent into the pressure isolated region.

### SUMMARY

Certain aspects, features and configurations are described below for various devices and methods that can be used to treat disorders in tissues.

In one aspect, a method of treating a skin lesion is provided. In certain examples, the method comprises increasing local pressure within a skin lesion by pressure isolation of the skin lesion, and intralesionally introducing a therapeutic agent into the pressure isolated skin lesion, in which the therapeutic agent is introduced in at least an effective amount to treat the skin lesion.

In certain instances, the method comprises clamping the skin lesion to pressure isolate the skin lesion. In other instances, the method comprises providing hydraulic pressure to the skin lesion to pressure isolate the skin lesion. In some configurations, the hydraulic pressure is a positive hydraulic pressure. In other examples, the method comprises providing pneumatic pressure to the skin lesion to pressure isolate the skin lesion. In certain instances, the pneumatic pressure is a negative pneumatic pressure. In some embodiments, the introducing step comprises introducing the therapeutic agent in a treatment volume, and in which the method further comprises removing about a same volume as the treatment volume from the skin lesion after a first treatment period. In other instances, the method comprises reintroducing the intralesionally introduced therapeutic agent for a second treatment period after the first treatment period. In some instances, the method comprises selecting the therapeutic agent to be an agent that is systemically toxic. In other instances, the method comprises introducing the therapeutic agent into the pressure isolated skin lesion in a concentration that is at least 10 times greater than a systemically toxic concentration used for the therapeutic agent.

In other embodiments, the method comprises intralesionally introducing a second therapeutic agent different from the first therapeutic agent. In some examples, the skin lesion comprises basal cell carcinoma and the therapeutic agent is bleomycin. In additional examples, the bleomycin is introduced into the skin lesion at a concentration at least 10 times greater than a systemically toxic concentration of bleomycin. In some configurations, the pressure isolation step comprises clamping the skin lesion between a first mechanical pressure member and a second mechanical pressure member. In other configurations, the pressure isolation step comprises providing a positive pressure by engaging the skin lesion with a first mechanical pressure member. In further configurations, the pressure isolation step comprises providing a negative pressure using a negative pneumatic pressure. In some embodiments, the negative pneumatic pressure is provided by drawing the skin lesion into a vacuum device to pressure isolate the skin lesion. In additional embodiments, the pressure isolation step comprises providing a positive pressure by engaging the skin lesion with a first pressure member, in which the first pressure member is configured to expand upon application of a hydraulic force to pressure isolate the skin lesion. In some instances, the pressure isolation step comprises providing a negative pressure by engaging the skin lesion with a first pressure member, in which the first pressure member is configured to contract upon application of a negative hydraulic force to pressure isolate the skin lesion. In other examples, the skin lesion comprises basal cell carcinoma and the therapeutic agent is bleomycin, in which the bleomycin is introduced into the skin lesion at a concentration at least 10 times greater than a systemically toxic concentration of bleomycin, and in which the introduced bleomycin is substantially removed from the skin lesion after a first treatment period.

In another aspect, a method of treating a disorder in an elastic tissue, the method comprising pressure isolating a region of the elastic tissue comprising the disorder, introducing a therapeutic agent in a treatment volume into the pressure isolated region, in which the therapeutic agent is introduced in at least an effective amount to treat the disorder, and removing substantially all of the treatment volume introduced into the pressure isolated region is disclosed.

In certain embodiments, the method comprises releasing the pressure of the pressure isolated region. In other embodiments, the method comprises pressure isolating the region after the pressure has been released. In other examples, the method comprises introducing an additional therapeutic agent into the pressure isolated region. In some embodiments, the method comprises configuring the therapeutic agent and the additional therapeutic agent as to be the same therapeutic agent. In additional examples, the method comprises providing hydraulic pressure to the region to pressure isolate the region. In some embodiments, the hydraulic pressure is configured to be a positive hydraulic pressure. In other embodiments, the method comprises providing pneumatic pressure to the region to pressure isolate the region. In some instances, the method comprises configuring the pneumatic pressure to be a negative pneumatic pressure. In certain examples, the method comprises selecting the therapeutic agent to be an agent that is systemically toxic. In some embodiments, the method comprises introducing the therapeutic agent into the pressure isolated region in a concentration that is at least 10 times greater than a systemically toxic concentration used for the therapeutic agent. In certain instances, the region comprises basal cell carcinoma and the therapeutic agent is bleomycin. In some embodiments, the bleomycin is introduced into the region at a concentration at least 10 times greater than a systemically toxic concentration of bleomycin.

In certain embodiments, the pressure isolation step comprises clamping the region between a first mechanical pressure member and a second mechanical pressure member. In some examples, the pressure isolation step comprises providing a positive pressure by engaging the region with a first mechanical pressure member. In other embodiments, the pressure isolation step comprises providing a negative pressure using a negative pneumatic pressure. In certain examples, the negative pneumatic pressure is provided by drawing the region into a vacuum device to pressure isolate the region. In other examples, the pressure isolation step comprises providing a positive pressure by engaging the region with a first pressure member, in which the first pressure member is configured to expand upon application of a hydraulic force to pressure isolate the region. In some examples, the pressure isolation step comprises providing a negative pressure by engaging the region with a first pressure member, in which the first pressure member is configured to contract upon application of a negative hydraulic force to pressure isolate the region. In other embodiments, the region comprises basal cell carcinoma and the therapeutic agent in the treatment volume is bleomycin, in which the bleomycin is introduced into the region at a concentration at least 10 times greater than a systemically toxic concentration of bleomycin, and in which the introduced treatment volume is substantially removed from the region after a first treatment period.

In an additional aspect, a device configured to reversibly couple to a tissue site to treat a tissue disorder at the tissue site is described. In some instances, the device comprises at least one pressure member configured to increase the local pressure within a region of the tissue site to pressure isolate the region and reduce blood or fluid flow into and out of the pressure isolated region, the device further configured to permit introduction of a therapeutic agent in a treatment volume into the pressure isolated region for a treatment period and to permit removal of substantially all of the treatment volume from the pressure isolated region after the treatment period.

In certain configurations, the pressure member is configured as a passive member. In other configurations, the device comprises at least one second pressure member configured to cooperate with the pressure member to provide the pressure isolation. In some examples, the second pressure member and the pressure member are configured to isolate the region by positioning the region between the second pressure member and the pressure member. In other examples, the pressure member is configured as a mechanical pressure member. In some embodiments, the pressure member is configured as a hydraulic pressure member. In further embodiments, the pressure member is configured as a pneumatic pressure member. In additional embodiments, the device further comprises fluid means configured to introduce the treatment volume into the pressure isolated region. In some examples, the device further comprises a fluid reservoir configured to retain the therapeutic agent. In other examples, the device comprises temperature means configured to heat the pressure isolated region.

In another aspect, a system configured to reversibly couple to a tissue site to treat a tissue disorder is disclosed. In some configurations, the system comprises pressure means configured to pressure isolate a region of the tissue site, and introduction means configured to introduce an effective amount of a therapeutic agent in a treatment volume into the pressure isolated region to treat the tissue disorder.

In certain instances, the pressure means is configured to isolate the region by application of a hydraulic force to the pressure means. In other instances, the pressure means is configured to isolate the region by application of a negative pneumatic force using the pressure means. In some embodiments, the pressure means is configured to isolate the region by application of a positive pressure using a mechanical pressure member. In other examples, the introduction means comprises a needle and a syringe. In further embodiments, the pressure means comprises a plurality of pressure members configured in an array, in which each of the plurality of pressure members is configured to pressure isolate a sub-region of the region. In some examples, the introduction means is configured as a needle array with a respective needle for each of the plurality of pressure members. In other examples, the system comprises a fluid reservoir fluidically coupled to each of the needles, the fluid reservoir configured to provide the therapeutic agent to the needles. In further embodiments, the introduction means is also configured to remove the treatment volume after a treatment period. In additional examples, the system comprises a syringe fluidically coupled to the introduction means.

In another aspect, a method of treating a mass within or on an organ is provided. In certain examples, the method comprises increasing local pressure at the mass by pressure isolation of the mass from surrounding tissue, and introducing a treatment volume comprising a therapeutic agent into the pressure isolated mass, in which the therapeutic agent is introduced in an effective amount into the isolated mass to reduce the size of the mass within the organ.

In some examples, the method comprises removing the introduced treatment volume after a treatment period. In other examples, the method comprises clamping the mass to pressure isolate the mass. In some embodiments, the method comprises providing hydraulic pressure to the mass to pressure isolate the mass. In additional examples, the method comprises configuring the hydraulic pressure to be a positive hydraulic pressure. In further examples, the method comprises providing pneumatic pressure to the mass to pressure isolate the mass. In some embodiments, the method comprises configuring the pneumatic pressure to be a negative pneumatic pressure. In additional examples, the method comprises reintroducing the therapeutic agent for a second treatment period after the treatment period. In some configurations, the method comprises selecting the therapeutic agent to be an agent that is systemically toxic. In certain embodiments, the method comprises introducing the therapeutic agent into the pressure isolated skin lesion in a concentration that is at least 10, 20, 30, 40, 50 or even at least 100 times greater than a systemically toxic concentration used for the therapeutic agent.

In an additional aspect, a kit configured to treat a tissue disorder at a tissue site is provided. In certain configurations, the kit comprises a pressure member configured to pressure isolate a region of the tissue site comprising the tissue disorder, and instructions for using the pressure member to pressure isolate the region.

In some embodiments, the kit comprises introduction means configured to introduce a therapeutic agent into the pressure isolated region. In certain embodiments, the introduction means comprises a syringe. In other examples, the kit comprises a therapeutic agent. In some configurations, the pressure member is configured to provide pressure isolation by way of mechanical force. In other embodiments, the pressure member is configured to provide pressure isolation by way of hydraulic force. In some instances, the pressure member is configured to provide pressure isolation by way of pneumatic force. In additional examples, the pressure member is configured as a pressure member array. In some examples, the kit comprises a second pressure member, e.g., a second pressure member configured the same as or different than the pressure member.

Additional aspects, configurations, embodiments and examples are described below.

### BRIEF DESCRIPTION OF THE SEVERAL VIEW OF THE DRAWINGS

Certain specific aspects, embodiments and configurations are described with reference to the accompanying figures in which:

FIG. 1 is a block diagram showing an illustrative treatment protocol, in accordance with certain examples;

FIG. 2 is a block diagram showing another illustrative treatment protocol, in accordance with certain examples;

FIG. 3 is a block diagram showing an illustrative treatment protocol using perfusion, in accordance with certain examples;

FIGS. 4A and 4B are illustrations of a device configured to apply an external pressure, in accordance with certain configurations;

FIG. 5 is an illustration of a mechanical pressure member, in accordance with certain examples;

FIGS. 6-8 are illustration of different types of pressure devices, in accordance with certain embodiments;

FIG. 9A is a bottom view of a mechanical pressure member array, in accordance with certain examples;

FIG. 9B is a side view of a mechanical pressure member array, in accordance with certain examples;

FIG. 10 is an illustration of a hydraulic pressure member, in accordance with certain configurations;

FIG. 11 is an illustration of an array comprising three hydraulic pressure members, in accordance with certain configurations;

FIGS. 12A and 12B are illustration showing the use of a pneumatic pressure member, in accordance with certain examples;

FIG. 13 is an illustration of a pneumatic pressure member array, in accordance with certain embodiments;

FIGS. 14A and 14B are photographs showing a subject with basal cell carcinoma before (14A) and after (14B) treatment, in accordance with certain examples;

FIGS. 15A and 15B are histology specimens showing the results of a punch biopsy before (15A) and after (15B) treatment, in accordance with certain examples; and

FIGS. 16-19 are photographs showing a superficial basal cell carcinoma and its treatment, in accordance with certain examples.

### DETAILED DESCRIPTION

In certain configurations, the devices described herein can be configured to isolate one or more tissues, masses within tissue or other areas of the body using an applied external pressure. For example, mechanical devices can be used to isolate skin lesions or skin regions comprising a skin condition by applying an external pressure, e.g., mechanical, pneumatic and/or hydraulic pressure, suction, etc. to the lesion and/or an area surrounding the lesion. One or more therapeutic agents can then be introduced into an isolated lesion or region in an effective amount to treat the lesion or condition. In certain instances, isolation of the lesion permits introduction of the therapeutic agent into the isolated lesion or region in an amount that far exceeds the maximum safe concentration for systemic use, e.g., 10×, 20×, 30×, 40×, 50× or 100× more than the maximum safe concentration for systemic use. In some instances, the concentration of therapeutic introduced into the isolated lesion or region may be systemically toxic if introduced by way of oral, intravenous or intra-arterial means. As described in more detail herein, by isolating the tissue area using the applied external pressure, an introduced treatment volume comprising a therapeutic agent can be substantially contained within the tissue area and not perfuse into systemic circulation to any substantial degree. Various illustrative tissue types are described and other soft tissues or hard tissue disorders can be treated using the methods and devices described herein.

In certain embodiments, the agent introduced into the isolated region can be introduced by injection and subsequent removal (after a treatment period, e.g., 10-30 minutes), can be continuously introduced and removed, e.g., using a fluid circuit, can be introduced sequentially followed by removal after a treatment period, etc. In some instances, different concentrations of agent can be introduced at different times during a treatment period to enhance treatment. While the exact treatment period can vary, illustrative treatment periods include, but are not limited to, 10-30 minutes, though longer or shorter treatment periods may be used depending on the agent used and the concentration of agent introduced into the isolated region.

In certain embodiments, the agent can be introduced into the area from the top, at the side, from underneath or at other areas of the region to be treated. For example, the agent can be introduced from the side of a pressure isolated region underneath the pressure member. In an alternative configuration, the agent can be introduced through the pressure member and into a top surface of the isolated region. In some instances, a fluid circuit may be configured to introduce agent from one surface of the region and remove agent from another surface of the region. For example, the agent can be introduced through a top surface of the isolated region, e.g., using a needle, and removed from underneath the region, e.g., using a needle inserted into the base of the region from the side. If desired, any injection and/or removal angle can be used to introduce and remove agent from the isolated region.

In some examples, the agent can be introduced with one or more enzymes, excipients, buffers, salts, etc. to enhance delivery of the agent. For example, introduction of an enzyme such as hyaluronidase, pepsin, carboxypeptidase, etc. may be introduced prior to introduction of the agent, with the agent or after the agent to enhance treatment. In some embodiments, it may be desirable to introduce an enzyme into the region prior to introduction of agent to permit the agent to perfuse into the region easier.

In certain configurations of the devices and methods described herein, the devices and methods are passive in that no electric current is applied to drive the agent into the isolated region. For example, various regions can be isolated by application of external pressure and then an agent can be introduced into the isolated region without the use of any electric current, electric field or other electric force that may be applied using methods such as electrokinesis, electroporation or iontophoresis. If desired, however, the regions can be isolated by application of external pressure and agent can be driven into the isolated region using active technologies such as electrokinesis, electroporation, iontophoresis or other suitable techniques that use an electric current or an electric field. In some embodiments, heat, an electric field, a magnetic field or other external stimulus can be applied within the isolated area or external to the isolated area to enhance treatment of the area. The pressure member itself may comprise a suitable device to generate an electric field, magnetic field, apply heat, sound waves, etc. For example, an end of the pressure member that contacts the region to be isolated may comprise suitable devices and components to generate an electric field or other external fields or stimulus.

Certain configurations described herein refer to a pressure member. The pressure member can be used by itself, in combination with another pressure member (which may be the same or may be different) or in combination with a plurality of pressure members. Where a plurality of pressure members are used, the pressure members can be arranged in an array, e.g., a linear array or an “m” by “n” array where m and n are individually selected to be two to sixteen or more, e.g., a 2 by 2 array, 2 by 3 array or other array configurations can be used. Where an array is used, the number of members in the array can be selected, for example, to match the overall size of the tissue region to be treated.

In certain instances, the devices and methods described herein can apply a positive pressure to the area of the tissue to be pressure isolated, e.g., a downward pressure or pressure toward the area to be isolated. In other instances, the devices and methods described herein can apply a negative pressure to the area of the tissue to be pressure isolated, e.g., a pressure away from the area to be isolated. In other examples, both a positive pressure and a negative pressure can be applied. For example, a positive pressure can be first applied followed by a negative pressure (or vice versa) or both a positive and negative pressure can be applied simultaneously. As discussed in more detail below, the various pressures can be applied with mechanical devices, using hydraulic pressure, using pneumatic pressure or by other suitable pressure means.

In certain configurations, prior to application of pressure to localize a tissue or region thereof for treatment, it may be desirable to elongate the particular region by stretching the region to at least some degree. Stretching may permit for better introduction of therapeutic agent and can increase the internal pressure of the region as well. Stretching can be accomplished using the pressure members described herein or may be accomplished by manual stretching or elongation of the tissue prior to disposing a pressure member on the tissue region. In some instances, the region is stretched to thin the tissue but not stretched to such a degree that leads to tissue trauma or injury.

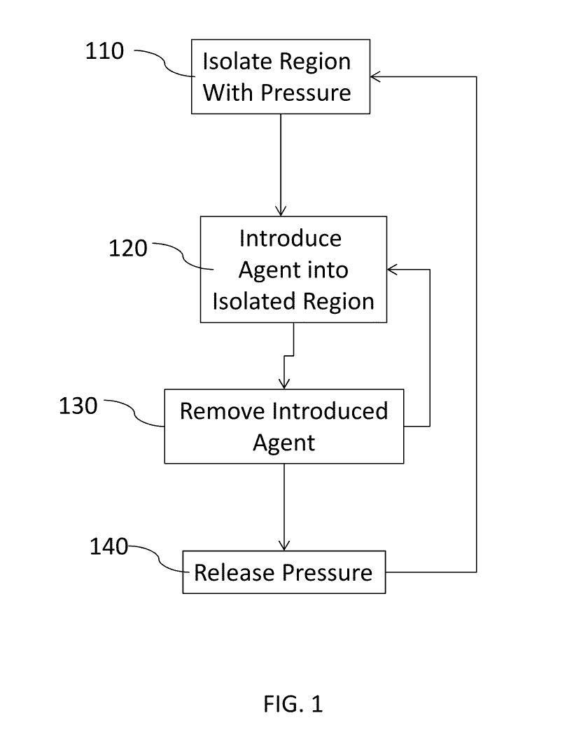

In certain embodiments and referring to FIG. 1, a block diagram showing some steps in a treatment protocol is shown. At a step 110, the lesion or region is isolated using pressure, e.g., mechanical, hydraulic or pneumatic pressure. As discussed in more detail herein, by isolating the lesion or region using pressure, flow into and out of the region can be reduced and/or cells within the isolated region may be rendered more permeable to uptake or certain species. In some instances, the region can be isolated for a sufficient period to effectuate treatment but not so long as to render the cells hypoxic or subject to conditions that would otherwise result in cell death. After isolation of the region, in a step 120, a therapeutic agent (in a treatment volume) can be introduced into the isolated region to treat a particular disorder or disease in that region. For example, where the isolated region comprises cancerous cells or tissues, an anti-cancer agent or anti-neoplastic agent can be introduced to treat the cancerous cells. The agent is typically permitted to remain resident within the isolated lesion or region for a treatment period. While the exact amount and type of agent introduced into the isolated region can vary, isolation of the region can prevent flow of the agent out of the isolated region in substantial amounts, which can permit introduction of very high concentrations, e.g., systemically toxic amounts, into the isolated region and/or can permit the use of therapeutic agents that are toxic systemically but effective locally. Subsequent to the treatment period, the treatment volume can be removed from the isolated region at a step 130. Removal can take place in many different manners as discussed in more detail below. In some instances, about 90% or more of the treatment volume is removed. As described in more detail herein, where the concentration of therapeutic agent used is substantially higher than that used in systemic concentrations, about 90% or more of the introduced agent is removed in the treatment volume. Once the treatment volume is removed from the isolated region, the pressure can be released at a step 150 to permit blood or other fluids to reenter the region under normal body pressure. In some instances, the steps 110-140 can be repeated two, three or more times to provide for sequential pressure isolation, agent introduction, agent removal and pressure release treatment of a particular lesion or region. In some instances, the concentration of therapeutic agent introduced into the region exceeds the systemically toxic concentration of the agent in that particular mammal, e.g., exceeds the systemically toxic amount in a human. For example, by isolating the region using pressure, the introduced agent is kept substantially within the isolated region and agent toxicity to organs, e.g., heart, liver, kidney, pancreas, etc., is mitigated. It is a substantial attribute that systemically toxic concentrations of therapeutic agent can be introduced into isolated regions without inducing overall toxic effects to the subject.

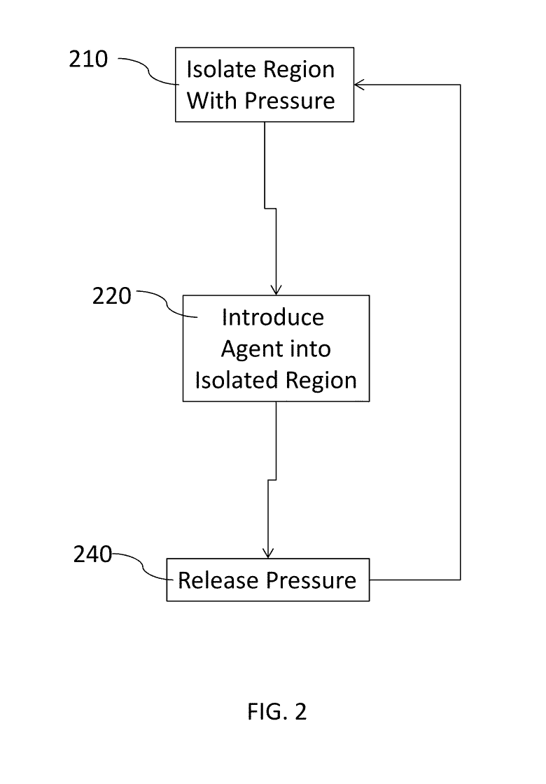

In other instances it may be desirable or feasible to permit release of the introduced agent after introduction. Referring to FIG. 2, in a step 210, the lesion or region can be isolated using pressure. An agent is then introduced into the isolated region at a step 220. The agent may remain resident in the isolated region for a desired period, e.g., a treatment period. The pressure can then be released to permit the introduced agent to move out of the region and/or to permit blood, oxygen or other fluids to move into the region. This process can be repeated a desired number of times, e.g., two, three, four or more. Where the agent is permitted to move into the systemic circulation of a subject, the amount of agent introduced can be selected such that it is not toxic to the subject. Introduction into the isolated region, however, can permit introduction of locally higher concentrations than might be feasible by oral, intravenous or other introduction routes. For example, the local concentration of the agent is dramatically increased (for at least some period) by introducing the agent into a pressure isolated region. This increase in concentration permits increased uptake by cells within the region, e.g., cancerous cells or other abnormal cells within the isolated region.

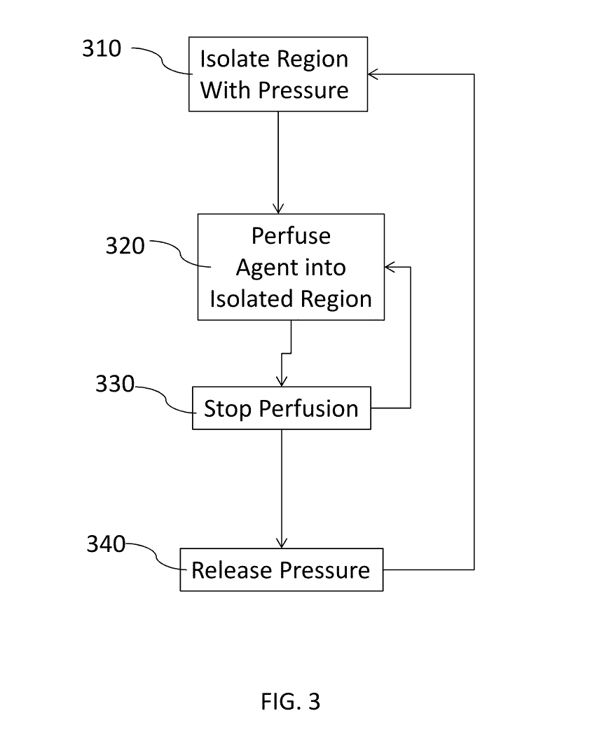

In certain configurations, the agent can be introduced into an isolated region by way of perfusion. For example, a fluid circuit can be used to couple the pressure isolated region to a reservoir of the agent. The fluid circuit permits continuous introduction and removal of the agent during the treatment. In some instances, application of a pressure can act to create flow through the isolated region. For example, suction (or other means) can be applied to create fluid flow in during the treatment. Referring to FIG. 3, a treatment protocol is showing using perfusion of the agent. In certain embodiments, at a step 310, the region is isolated with pressure. A fluid circuit is then fluidically coupled to the region to permit perfusion of the agent into and out of the isolated region at a step 320. Initially, the pressure used to introduce the agent can be less than the pressure used to remove the agent to permit the agent and fluid to accumulate, at least to some degree, in the isolated region. The perfusion medium can include the agent, oxygenated buffer or other species, gases or fluids to assist in introduction of the agent and/or in keeping any cells within the isolated region alive. Agent can be perfused for a desired period, e.g., a treatment period, and the perfusion can be stopped at a step 330. In some instances, the pressure used to remove the agent is applied for a certain time to ensure at least about 90% of the treatment volume has been removed from the isolated region prior to stopping perfusion. The pressure can then be released at a step 340 to permit blood, oxygen or other species to enter into the region. In some instances, the perfusion pressure applied is not so high as to disrupt cell membranes but is high enough to ensure that the agent is forced into contact with the cells of the isolated region. For example, isolation of the region using pressure provides a certain internal pressure within the isolated region. The perfusion pressure typically exceeds the pressure of the isolated region so that agent flows into the region down the pressure gradient. The perfusion pressure need not be constant during treatment and may increase or decrease as desired. If desired and prior to release of the pressure, one or more additional fluids with or without the agent or with a different agent can be introduced into the isolated region.

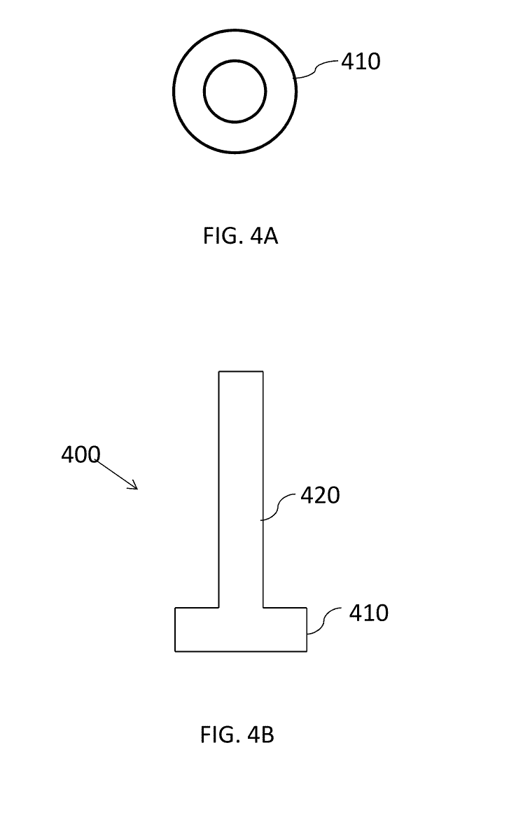

In certain configurations, the pressure application described herein can be provided with many different types of devices. For example, devices described herein can apply a suitable pressure to reduce perfusion levels into and/or out of the isolated region. Referring to FIGS. 4A and 4B, a device 400 is shown that comprises a generally annular base 410 coupled to a longitudinal shaft 420 positioned orthogonally to the base 410. In some instances, the opening of the base 410 can be selected to substantially encompass the region to be isolated. For example, the dimensions and shape of the base can be sized and arranged to mirror that of the region to be isolated. In some embodiments, the shaft 420 can be hollow, if desired, or may comprise one or more internal openings. In certain applications, the base 410 can be disposed onto the region to be pressure isolated, and mechanical pressure can be applied by forcing the base downward around the region. The device 400 generally is designed to apply the downward pressure from one side of the region, but devices that can apply pressure from two sides are described in more detail below. If desired, a support can be positioned on an opposite side of the region so that the region is pressed between the support and the base 410 of the device 400. In other instances, two of the device 400 can be used to isolate the region by placing the region between the two devices and application of a pressure toward the region to sandwich the region in the bases of the two devices. The external pressure can serve to isolate the region to at least some degree. In other instances, the region can be drawn into the opening of the base 410 by applying an external pressure, e.g., vacuum, to the surface of the region. The applied vacuum can enhance isolation of the region within the base 410. If desired, a sealing agent such as a grease or oil can be applied to the region prior to application of the device 400 to provide a seal between the region and the base 410 of the device 400. In some instances, the base 410 can be temperature controlled to cool and/or heat the isolated region for added benefit. For example, the base 410 may comprise a coolant material that is resistant to temperature changes during the treatment period.

In some configurations, the therapeutic agent can be introduced into the isolated region through the shaft 420 of the device 400. For example, the shaft 420 may be sized and arranged to permit insertion of a needle through the shaft and into the pressure isolated region, or the shaft itself may comprise an integral needle and/or syringe that can be used to introduce an agent into the isolated region. In some instances, the needle/syringe may be pre-loaded with agent at a desired amount such that substantially all agent in the syringe of the shaft is introduced to treat the isolated region by injection of a treatment volume in the syringe. Similarly, the volume of the syringe can be sized such that substantially all of the treatment volume can be removed post-introduction using the needle/syringe. In this manner, dosing of the isolated region with an agent can be simplified by integrating the pressure device with the agent delivery device. In other instances, the device 400 may be part of a fluid circuit that can perfuse agent into and out of the isolated region. For example, the isolated region may be isolated and present within the opening of the base 410 and fluid can be introduced and/or removed using suitable fluid lines placed through the longitudinal shaft 420 of the device 400 and/or through holes in the skin. A fluid circuit can introduce and remove agent into and out of, respectively, the pressure isolated region.

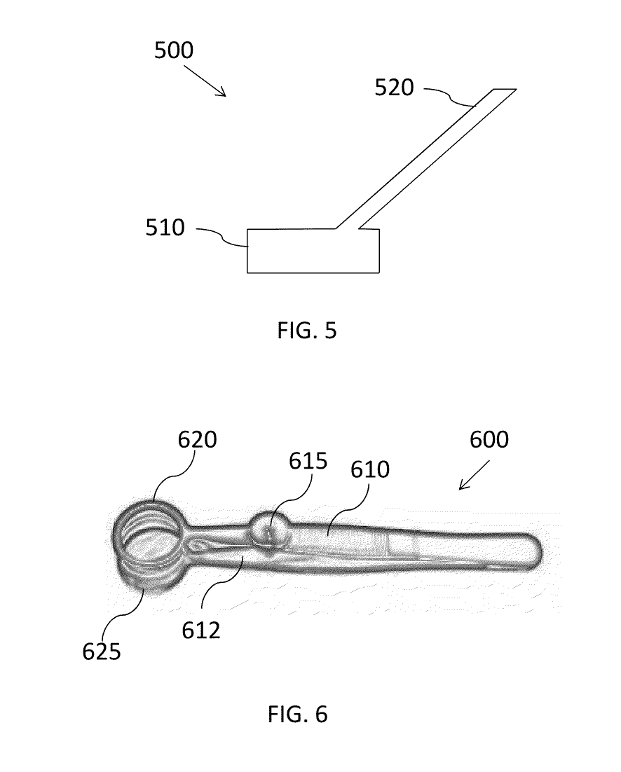

In certain embodiments, the shaft of the pressure device can be angled at less than 90 degrees to the base while still permitting the base to be open to the environment, e.g., while still permitting additional pressure to be applied to the isolated region using a vacuum. Referring to FIG. 5, a device 500 is shown that comprises a base 510 coupled to an angled shaft 520. The base 510 may comprise an opening similar to the one shown for base 410, e.g., an opening sized and arranged to permit location of a region within the inner opening of the base 510. In some instances, the base 520 can be attached to the shaft 510 through a moveable joint (not shown) to permit an end user to adjust the overall angle between the shaft 510 and the base 520. The base 510 may comprise an opening to permit a user to introduce a therapeutic agent into an isolated region through the opening as described herein, e.g., using a needle/syringe or a fluid circuit as described herein.

In certain configurations, a device suitable for isolating a region and permitting introduction of a therapeutic agent into the isolated region may comprise an open surface and a closed surface that together are operative to isolate the region. Referring to FIG. 6, a device 600 is shown comprising arms 610, 612 that are configured to be pushed toward each other. A retention mechanism 615 is shown that can be used to hold the arms 610, 612 a desired spacing from each other. The device 600 also comprises a first pressure member 620 and a second pressure member 625. The first pressure member 620 is annular in shape and comprises a central opening. The second pressure member 625 is also annular in shape but is closed or does not include any central opening. In use of the device 600, a tissue is placed between the pressure members 620, 625 and sufficient force is applied to the arms 610, 612 to pressure isolate that region of the tissue. The retention mechanism 615 is then tightened to maintain the applied external pressure. A therapeutic agent (in a treatment volume) can then be introduced into the isolated region through the use of a needle/syringe, perfusion by way of a fluid circuit or through other means. Similarly, the treatment volume can be removed after a treatment period using the same or similar devices. Pressure can be released on the isolated region by loosening the retention mechanism 615 to permit blood and/or other fluid to flow into the previously isolated region. This process can be repeated a desired number of times, and the agent used in successive applications may be the same or may be different. Illustrative tissue suitable for use with the device 600 may include, but it not limited to, skin, lung tissue, bladder tissue, gastrointestinal tissues (e.g., esophagus, stomach, small intestine, large intestine), kidney tissue, breast tissue, prostate tissue, pancreas, ovary and other tissues that can stretch or are elastic to at least some degree, e.g., can be stretched without failure or permanent trauma caused by application of external pressure. In other instances, reduced pressure can be used in less elastic tissues, e.g., muscle, cartilage, etc., to pressure isolate these regions without causing permanent damage or trauma.

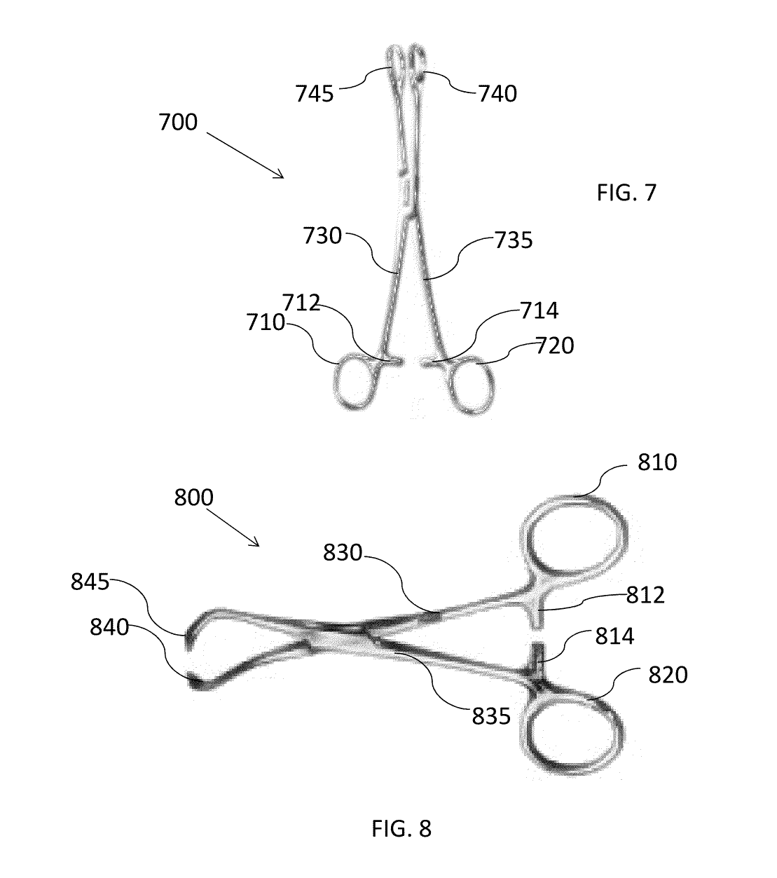

In some configurations, a device suitable for isolating a region and permitting introduction of a therapeutic agent into the isolated region may comprise an first open surface and a second open surface that together are operative to isolate the region. Referring to FIG. 7, a device 700 is shown comprising finger inserts 710, 720 that are configured to be pushed toward each other. An arm 730 comprises a first retention mechanism 712, and an arm 735 comprises a second retention mechanism 714. The arm 730 also comprises a first open pressure member 740, and the arm 735 comprises a second open pressure member 745. Each of the pressure members 740, 745 comprises a central opening and is generally annular or ring-shaped. In use of the device 700, a tissue is placed between the pressure members 740, 745 and sufficient force is applied to the finger openings 710, 720 to cause the distance between the two openings 710, 720 to narrow. At some point, the retention mechanisms 712, 714 engage each other and lock the pressure members 740, 745 into a fixed position and at a fixed applied pressure to the tissue between the members 740, 745. A therapeutic agent (in a treatment volume) can then be introduced into the isolated region through the use of a needle/syringe, perfusion by way of a fluid circuit or through other means. The treatment volume can be removed after a treatment period using the same or similar devices. Pressure can be released on the isolated region by disengaging the retention mechanisms 712, 714 to permit blood and/or other fluid to flow into the previously isolated region. This process can be repeated a desired number of times, and the agent used in successive applications may be the same or may be different. Illustrative tissue suitable for use with the device 700 may include, but it not limited to, skin, lung tissue, bladder tissue, gastrointestinal tissues (e.g., esophagus, stomach, small intestine, large intestine), kidney tissue, breast tissue, prostate tissue, pancreas, ovary and other tissues that can stretch or are elastic to at least some degree, e.g., can be stretched without failure or permanent trauma caused by application of external pressure. In other instances, reduced pressure can be used in less elastic tissues, e.g., muscle, cartilage, etc., to isolate these regions without causing permanent damage or trauma.

In additional configurations, the pressure members used in the devices described herein need not be annular or ring shaped or have any specific overall surface area. If the area to be treated is small, e.g., where a mole, small skin lesion or other small areas is to be isolated, then the surface area of the pressure members can also be small. Referring to FIG. 8, a device 800 is shown that comprises finger openings 810, 820, retention members 812, 814 on arms 830, 840, respectively, and pressure members 840, 845. In use of the device 800, a tissue is placed between the pressure members 840, 845 and sufficient force is applied to the finger openings 810, 820 to cause the distance between the two openings 810, 820 to narrow. At some point, the retention mechanisms 812, 814 engage each other and lock the pressure members 840, 845 into a fixed position and at a fixed applied pressure to the tissue between the members 840, 845. In some instances, the isolated tissue may rest above the points of the pressure member 840, 845, and the agent (in a treatment volume) can be introduced into the isolated tissue at any desired angle. The treatment volume can be removed after a treatment period using the same or similar devices. Pressure can be released on the isolated region by disengaging the retention mechanisms 812, 814 to permit blood and/or other fluid to flow into the previously isolated region. This process can be repeated a desired number of times, and the agent used in successive applications may be the same or may be different. Illustrative tissue suitable for use with the device 800 may include, but it not limited to, small lesions or growths on the skin, lung tissue, bladder tissue, gastrointestinal tissues (e.g., esophagus, stomach, small intestine, large intestine), kidney tissue and other tissues that can stretch or are elastic to at least some degree, e.g., can be stretched without failure or permanent trauma caused by application of external pressure. In other instances, reduced pressure can be used in less elastic tissues, e.g., small lesions on muscle, cartilage, etc., to isolate these regions without causing permanent damage or trauma.

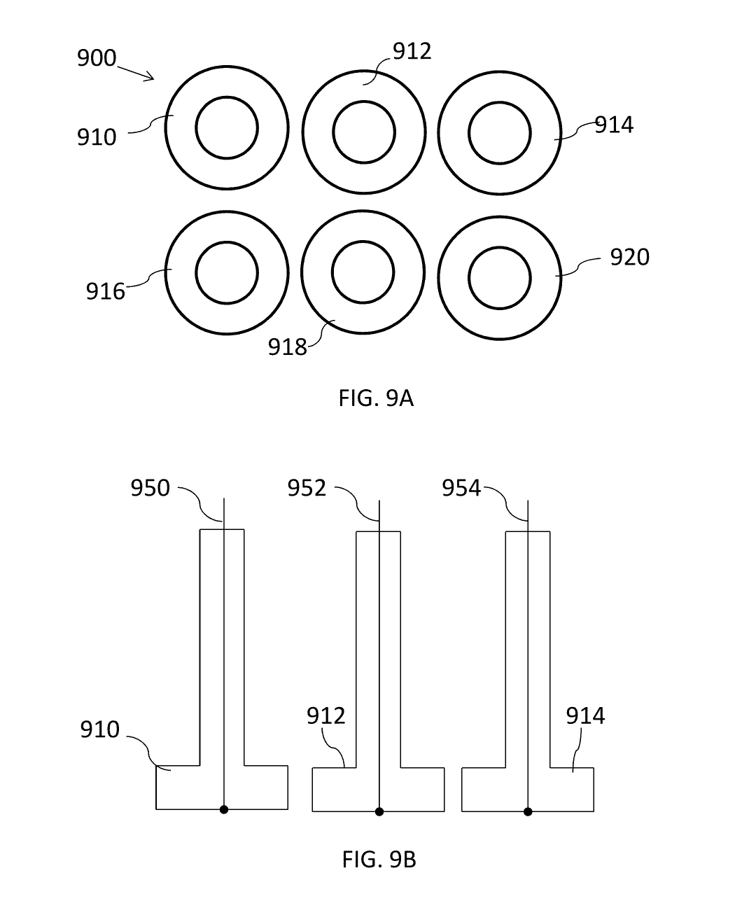

In certain configurations, it may be desirable to treat large areas using an array of mechanical pressure members. For example, a plurality of individual pressure members can be arranged together to permit a plurality of different regions to be pressure isolated. Each mechanical pressure member of the array can be configured as described herein, e.g., as described in connection with FIGS. 4A-8, to permit each member to pressure isolate a region of the tissue to be treated. Referring to FIG. 9A, a bottom view of an illustrative array 900 is shown where a plurality of pressure members 910, 912, 914, 916, 918 and 920 are arranged beside each other. In the array 900, the pressure members are configured similar to that shown in FIGS. 4A and 4B, but if desired, each of the pressure members 910-920 can instead be designed to clamp a region of the tissue. By using the array 900, different regions of a large tissue site to be treated can be pressure isolated to permit introduction of a therapeutic agent into each of the pressure isolated regions. For example, the array 900 can pressure isolate the various regions, and a therapeutic agent (in a treatment volume) can be introduced into each of the pressure isolated regions. The treatment volume can then be removed after a treatment period from each of the regions using the same or similar devices used to introduce the agent, e.g., using a needle/syringe. Pressure can be released on the isolated regions by disengaging the pressure members 910-920 to permit blood and/or other fluid to flow into the previously isolated regions. In some instances, it may be desirable to use a respective needle/syringe for each member of the array. A side view is shown in FIG. 9B of an array comprising three mechanical pressure members 910, 912 and 914 each including a respective needle 950, 952 and 954. The needles can be used to introduce an agent into each of the regions within the pressure members. In some instances, the same therapeutic agent can be introduced into each pressure isolated region. For example, each needle can be fluidically coupled to a common syringe or reservoir that can provide a treatment volume comprising the therapeutic agent to each of the pressure isolated regions. In other instances, different therapeutic agents can be introduced into different areas. In additional configurations, all regions may receive the same therapeutic agent but the therapeutic agent can be introduced or removed at different times, e.g., the treatment period of different regions can vary. After the therapeutic agent remains in the area for a treatment period, about the same volume as the introduced treatment volume can then be removed using the needle. The pressure can then be released on the regions to permit blood or fluid to flow back into those regions under normal pressure. The exact number of mechanical pressure members in the array may vary, for example, from about two to about sixteen or more. It may be desirable to use fewer mechanical pressure members where smaller regions are to be treated and to use more mechanical pressure members where larger regions are to be treated.

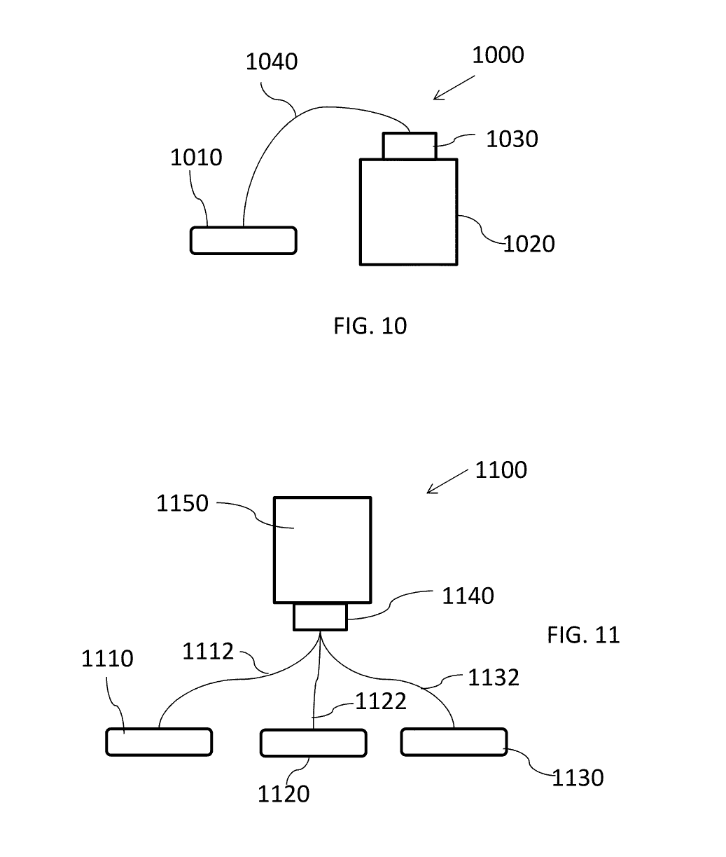

In certain embodiments, the pressure devices described herein can use a liquid to provide a hydraulic pressure at a specific tissue site or region. For example, a device can be placed over the area to be treated, and a hydraulic pressure can be applied to the device to cause expansion of the device and force the device downward toward the area to be pressure isolated. This forcing results in slowing or reduction of blood or other fluid into the region within the pressure member. In some instances, the pressure member can be produced from elastomeric materials that can expand to at least some degree to provide for added pressure with an increase in the hydraulic pressure. Referring to FIG. 10, a pressure device 1000 comprises an expandable pressure member 1010 fluidically coupled to a hydraulic fluid reservoir 1020 and to a pump 1030 through a fluid line 1040. If desired, however, the fluid line 1040 may instead be coupled to a syringe (not shown) to permit a user to increase the pressure provided by the pressure member 1010, e.g., by depression of the syringe plunger to force hydraulic fluid from the syringe and into the pressure member 1010 and fluid line 1040. Where a pump 1030 and fluid reservoir 1020 are used, a pressure controller can be present to provide a set pressure to the pressure isolated region. The pressure member 1010 can be configured similar to other pressure members described herein, e.g., may comprise a central opening to permit introduction of an agent into the pressure isolated region. In some instances, a therapeutic agent (in a treatment volume) can then be introduced into the isolated region through the use of a needle/syringe, perfusion by way of a fluid circuit or through other means. The treatment volume can be removed after a treatment period using the same or similar devices. Pressure can be released on the isolated region by pumping fluid back into the fluid reservoir 1020 to permit contraction of the pressure member 1010. If desired, the pressure member 1010 may be disposed on the tissue or region using a sealant or other material that can hold the pressure member in place for at least some period.

In some embodiments, a device similar to the device 1000 of FIG. 10 can be used to apply a negative pressure to a region to be pressure isolated. For example, hydraulic fluid within the pressure member 1010 can be removed to result in contraction of the member 1010. Tissue within any interior open space of the pressure member 1010 can be pulled inward as the pressure member 1010 is pulled inward from the reduction in hydraulic pressure. Where the pressure member 1010 is disposed on the tissue region using a sealant, movement of the pressure member 1010 causes similar movement of the tissue. Once the tissue is pulled into the pressure member, a therapeutic agent (in a treatment volume) can then be introduced into the pressure isolated region through the use of a needle/syringe, perfusion by way of a fluid circuit or through other means. The treatment volume can be removed after a treatment period using the same or similar devices. Pressure can be restored to the isolated region by pumping fluid back into the pressure member 1010 to permit expansion of the pressure member 1010.

In certain instances, the pressure member 1010 may first be expanded using increased hydraulic pressure, e.g., to stretch the region to be isolated, and then the pressure member 1010 can be contracted to draw the tissue upward into the pressure member 1010. This process of expansion and contraction can be repeated a desired number of times. A therapeutic agent can then be introduced into the pressure isolated region through the use of a needle/syringe, perfusion by way of a fluid circuit or through other means. The introduced agent can be removed after a treatment period using the same or similar devices. Pressure can be restored to the isolated region by pumping fluid back into the pressure member 1010 to permit expansion of the pressure member 1010. Similarly, it may be desirable to first draw the region to be treated into the pressure member 1010 by first applying a negative pressure to force contraction of the pressure member 1010. Then, positive pressure can be provided to force the pressure member 910 to expand. This process can be repeated a desired number of times. Once the pressure member 910 is in the expanded state, a therapeutic agent (in a treatment volume) can then be introduced into the pressure isolated region through the use of a needle/syringe, perfusion by way of a fluid circuit or through other means. The treatment volume can be removed after a treatment period using the same or similar devices. Pressure can be restored to the isolated region by pumping fluid back into the fluid reservoir 1020 to permit contraction of the pressure member 1010.

In certain embodiments, the hydraulic pressure members described herein can be positioned in an array to permit pressure isolation of a plurality of regions on the tissue. For example and referring to FIG. 11, a plurality of hydraulic pressure members 1110, 1120 and 1130 can be used where each hydraulic pressure member is configured to pressure isolate a particular region of the tissue to be treated. If desired, each hydraulic pressure member 1110, 1120, and 1130 may comprise a respective needle (not shown) to permit introduction of a therapeutic agent into the region that has been pressure isolated by that particular hydraulic pressure member. A fluid reservoir 1150 and pump 1140 can be coupled to each of the hydraulic pressure members 1110, 1120 and 1130 through a respective fluid line 1112, 1122 and 1132. In some instances, the same therapeutic agent can be introduced into each pressure isolated region. For example, each needle can be fluidically coupled to a common syringe or reservoir that can provide a treatment volume comprising the therapeutic agent to each of the pressure isolated regions. In other instances, different therapeutic agents can be introduced into different areas. In additional configurations, all regions may receive the same therapeutic agent but the therapeutic agent can be introduced or removed at different times, e.g., the treatment period of different regions can vary. After the therapeutic agent remains in the area for a treatment period, about the same volume as the introduced treatment volume can be then removed using the needle. The hydraulic pressure can then be released on the regions to permit blood or fluid to flow back into those regions under normal pressure. The exact number of hydraulic pressure members in the array may vary, for example, from about two to about sixteen or more. It may be desirable to use fewer hydraulic pressure members where smaller regions are to be treated and to use more hydraulic pressure members where larger regions are to be treated.

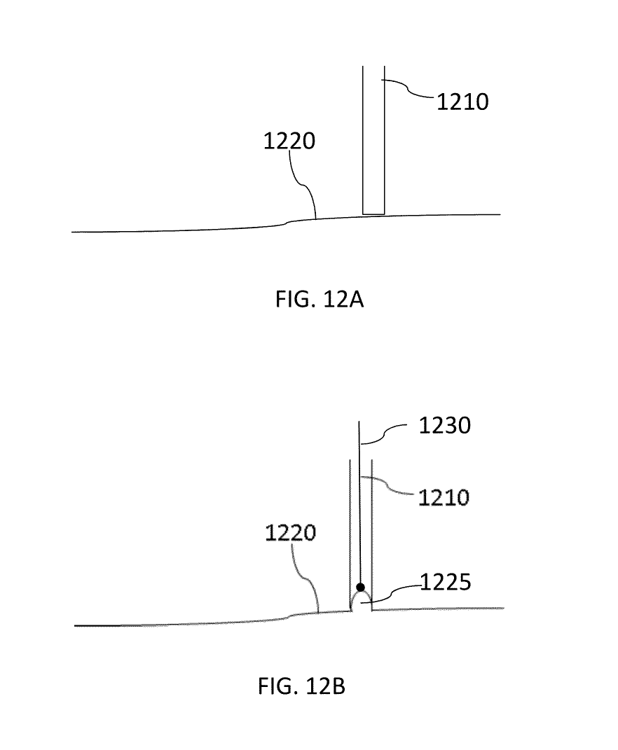

In some examples, pneumatic pressure can be used to provide a positive pressure or a negative pressure to the region to be pressure isolated. Referring to FIG. 12A, a pneumatic pressure member 1210 is shown positioned on a tissue surface 1220 in a first state or mode where substantially no pneumatic pressure is applied. In FIG. 12B, a negative pneumatic pressure has been applied to pull a region 1225 of the tissue surface 1220 into the pressure member 1210. A needle 1230 can be used to introduce a therapeutic agent into the pressure isolated region 1225. After the therapeutic agent remains in the region 1225 for a treatment period, about the same volume as the introduced treatment volume can be then removed from the region 1225 using the needle 1230. The pneumatic pressure can then be released on the region 1225 to permit blood or fluid to flow back into the region 1215 under normal pressure. The pressure applied in the example shown in FIGS. 12A and 12B is a negative pressure, e.g., vacuum pressure, which functions to suck the region into the pneumatic pressure member 1210. In other situations, a positive pneumatic pressure can be applied by providing a gas into the pressure member 1210. For example, the pressure member 1210 can be configured as a sleeve with an inner wall, an outer wall and space between the two walls, and a gas can be provided to the space to provide a ring of pressurized gas to the region to be pressure isolated. Other configurations that can provide a positive pneumatic pressure will be recognized by the person of ordinary skill in the art, given the benefit of this disclosure. After the region is sucked into the pressure member 1210, agent can be introduced into the region within the pressure member 1210, e.g., intermittently or through a fluid circuit as described herein. For example, agent can be introduced through the top and removed from the bottom of the region or vice versa or agent can be introduced and removed through the same site.

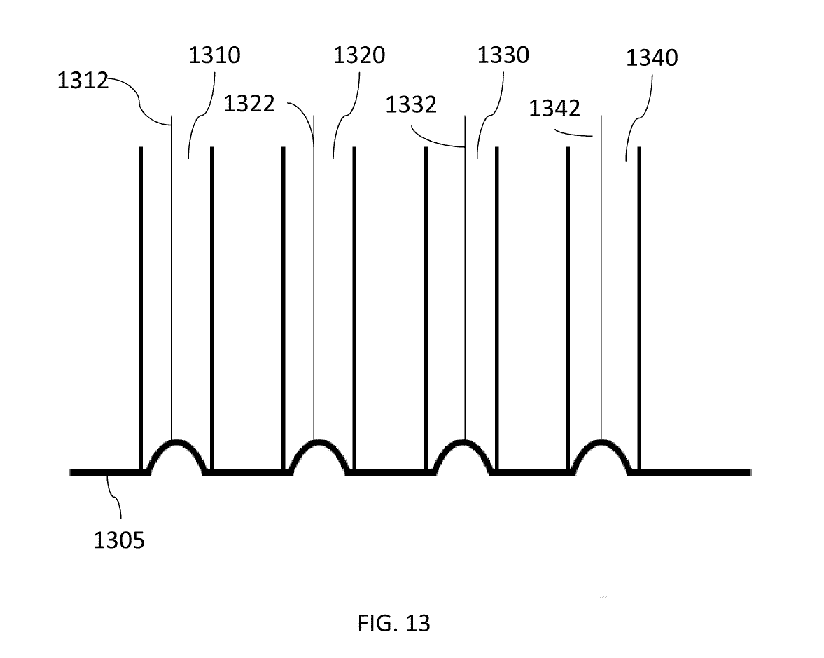

In some instances, an array of pneumatic pressure members can be used to pressure isolate a plurality of different regions of a tissue. Referring to FIG. 13, a side view of an array comprising a row of four pneumatic pressure members 1310, 1320, 1330 and 1340 is shown. Each member of the array provides a negative pneumatic pressure to draw a region of the tissue 1305 into the pneumatic member. Each of the pneumatic pressure members 1310, 1320, 1330 and 1340 also comprises a respective needle 1312, 1322, 1332 and 1342. In certain configurations, the same therapeutic agent can be introduced into each pressure isolated region. For example, each needle can be fluidically coupled to a common syringe or reservoir that can provide a treatment volume comprising the therapeutic agent to each of the pneumatic pressure isolated regions. In other instances, different therapeutic agents can be introduced into different areas. In additional configurations, all regions may receive the same therapeutic agent but the therapeutic agent can be introduced or removed at different times, e.g., the treatment period of different regions can vary. After the therapeutic agent remains in the area for a treatment period, about the same volume as the introduced treatment volume can be then removed using the needles. The pneumatic pressure can then be released on the regions to permit blood or fluid to flow back into those regions under normal pressure. The exact number of pneumatic pressure members in the array may vary, for example, from about two to about sixteen or more. It may be desirable to use fewer pneumatic pressure members where smaller regions are to be treated and to use more pneumatic pressure members where larger regions are to be treated.

While the exact tissue condition that can be treated using the devices described herein can vary, in some instances disorders of the skin are treated. For example, skin conditions treatable using the devices disclosed herein can vary, and illustrative conditions include but are not limited to, skin cancer such as, for example, melanoma, carcinoma, basal cell carcinoma or other types. Melanoma is generally characterized by abnormal cell growth of melanocytes. The abnormal cell growth is typically caused by exposure to ultraviolet radiation. While melanoma is often removed surgically, the devices and methods described herein can also be used to treat melanoma. In particular, the devices and methods described herein can be used in adjuvant therapy to deliver interferon to isolated regions comprising melanoma. By delivering the interferon directly to the melanoma site, it may be possible to avoid systemic side effects often encountered with high-dose interferon treatments. In other instances, the devices and methods described herein can be used to deliver an agent including, but not limited to, one or more of dicarbazine, temozolide or interleukin-2 to pressure isolated melanoma sites. In additional instances, the devices and methods described herein can be used to deliver a biological agent such as an antibody, e.g., a monoclonal antibody such as, for example, ipilimumab to a pressure isolated melanoma site. As noted herein, the ability to delivery agents at pressure isolated sites permits the use of much higher concentrations than typically used with systemic delivery of the same agents. In examples where melanoma regions are pressure isolated and treated with a chemical agent or a biological agent, the agent can be delivered at a concentration that is 2×, 3×, 4×, 5×, 10× or more above the concentration normally delivered in an oral, intravenous, or other methods commonly used to introduce an agent into a mammal such as a human. After the agent has remained in the pressure isolated melanoma site for a treatment period, a treatment volume can be removed from the melanoma site and pressure can be removed.

In other instances, the devices and methods described herein can be used to treat carcinoma of the skin. Carcinoma is generally characterized by the abnormal growth of epithelial cells of the skin or other tissues that line the inner or outer surfaces of the body. Carcinomas are common in the lung, breast, prostate, colon, rectum, pancreas, liver, kidneys, ovaries and skin. There are many different types of carcinomas including, but not limited to, basal cell carcinoma, squamous cell carcinoma, renal cell carcinoma, and ductal carcinoma. Basal cell carcinoma is the most common form of all cancers. Basal cell carcinoma often occurs in the skin and exhibits open sores, red patches or shiny bumps or scars. The area of the skin comprising the basal cell carcinoma can be pressure isolated. The isolated region may then receive one or more therapeutic agents (in a treatment volume) including but not limited to chemical agents such as 5-fluorouracil, imiquimod, vismodegib, bleomycin or other neoplastic agents. If desired, a biological agent such as a monoclonal antibody can also be delivered. The agent may remain in the lesion for a treatment period and then the treatment volume be removed subsequent to treatment and prior to pressure release of the isolated region. Squamous cell carcinoma develops in the thin, flat squamous cells of the epidermis. Squamous cells also occur in the digestive tract, lungs, lips, mouth, esophagus, lung, bladder, prostate, vagina, cervix and other organs. Squamous cell carcinoma exhibits nodules, sores or scaly patches on the skin. Treatment of squamous cell carcinoma can be accomplished using the devices and methods described herein. In particular, the area comprising the squamous cell carcinoma can be pressure isolated, and then an agent can be introduced into the pressure isolated regions. Illustrative agents to treat the squamous cell carcinoma include, but are not limited to, imiquimod, afamelanotide, bleomycin and other neoplastic agents. After a treatment period, then agent can then be removed (by removal of the treatment volume), and the pressure can be released. Renal cell carcinoma originates in the lining of the proximal convoluted tubule. Conventional treatment is partial or complete removal of the kidneys as the cancer is resistant to radiation therapy and chemotherapy. The devices and methods described herein can be used to isolate the affected regions. For example, one or more chemical agents such as sunitinib, temsirolimus, bevacizumab, sorafenib or a kinase inhibitor or an angiogenesis inhibitor, bleomycin and other neoplastic agents can be delivered to the pressure isolated region. In other instances, a biological agent such as interferon-alpha can be delivered to the pressure isolated region and removed post-treatment.

In certain embodiments, the devices and methods described herein can be used to treat breast cancer. For example, invasive ductal carcinoma (or invasive carcinoma of no special type) is the most common type of breast cancer. The cancer begins in the milk ducts and spreads to other breast tissues. Current therapy involves removal of the lumps by way of a lumpectomy or radical mastectomy. By using the devices and methods described herein, it may be possible to retain the breast tissue while at the same time treating the carcinoma. For example, various regions of the breast can be pressure isolated particularly those masses with a size of less than 4 cm in diameter. One or more agents can be delivered (in a treatment volume) to the pressure isolated regions for a treatment period. The treatment volume can then be removed, and the pressure can be released. For example, agents can be delivered to isolated breast tissue regions at concentrations that exceed the systemically toxic concentration of the agent. The agent can be perfused, e.g., using suction or other means, for a treatment period. Illustrative agents include but are not limited to, hormonal therapies such as tamoxifen, neoplastic agents such as bleomycin, antibodies such as trastuzumab and other chemical or biological agents used to treat carcinomas of the breast.

In certain instances, the devices and methods described herein can be used to treat warts of the skin. Warts can occur at any part of the skin but are often found on the hands, feet, and eyelids. Warts are typically caused by a viral infection, e.g., by one or more variants of the human papillomavirus. Warts are generally classified into different types or categories including common wart (raised wart with rough surface), flat wart (smooth flattened warts), filiform warts (finger-like warts), genital warts, mosaic warts (clustered plantar-type warts), periungual warts (clusters of warts around the nails) and plantar warts (hard wart usually found on the soles of the feet). Other types of warts also exist. Existing therapies tend to target the external surface of the wart, e.g., using various acids, liquid nitrogen or other topical species. Using the devices and methods described herein, it is possible to deliver agents into the localized tissue comprising the wart. For example, the wart can be pressure isolated. One or more agents (in a treatment volume) can then be introduced into the wart to kill the underlying virus. The treatment volume can then be removed after a treatment period, and the pressure can be released. Illustrative agents to treat warts include, but are not limited to, imiquimod, cantharidin, fluorouracil, bleomycin or other neoplastic agents or biological agents such as interferon.

In certain configurations, the devices and methods described herein can be used to treat hemangiomas. A hemangioma is characterized by an abnormal growth that is usually filled with blood vessels. Hemangiomas are often categorized into three types including capillary hemangioma (salmon patch, port-wine stain, strawberry angioma, yin rose patch), venous hemangioma and arterial hemangioma. The devices and methods described herein can be used to treat the hemangioma. For example, the hemangioma can be pressure isolated using one or more of the devices described herein. The pressure isolated hemangioma can receive an agent (in a treatment volume) for a treatment period, and then the treatment volume can be removed and the pressure can be released. This process can be repeated if desired. In some instances, the particular agent introduced into the pressure isolated hemangioma can be a corticosteroid, an angiogenesis inhibitor, a beta blocker (e.g., propranolol, timolol), vincristine, bleomycin or other neoplastic agents or a biological agent such as interferon.

In certain embodiments, the devices and methods described herein can be used to treat psoriasis. Psoriasis is mediated by the immune system and is characterized by red, scaly skin patches often accompanied by itching. Plaque psoriasis is the most common type of psoriasis and has white scaly patches on the epidermis. Psoriasis is often treated with systemic administration of drugs, but these drugs can have serious side effects including reduced immune response. Using the devices and methods described herein, various psoriatic regions can be treated without the need to resort to systemic drug administration. For example, psoriatic regions can be pressure isolated. The pressure isolated regions can receive a therapeutic agent (in a treatment volume) for a treatment period. The treatment volume can then be removed, and the pressure can be released. Illustrative agents that can be introduced into pressure isolated psoriatic regions include, but are not limited to, methotrexate, ciclosporin, dimethyl fumarate, a retinoid, hydroxycarbamide, eicosapentaenoic acid, docosahexaenoic acid, or a biological agent such as infliximab, adalimumab, golimumab, certolizumab pegol, or etanercept or other biological or chemical agents can be introduced. In instances where a biological agent is introduced, it may not be desirable to remove the biological agent post-injection. Instead, the biological agent may be permitted to circulate systemically, if desired. In some instances, the devices and methods described herein may be co-administered with phototherapy and/or topical agents to provide for enhanced treatment of psoriasis.

In some embodiments, acne can be treated using the devices and methods described herein. Acne is typically characterized by areas of red skin, pimples and other skin discoloration and abnormalities. Using the devices and methods described herein, acne can be treated without the need to resort to systemic drug administration or topical application of agents. For example, acne regions, e.g., pimples, can be pressure isolated. The pressure isolated regions can receive a therapeutic agent (in a treatment volume) for a treatment period. The treatment volume can then be removed, and the pressure can be released. Illustrative agents that can be introduced into pressure isolated acne regions include, but are not limited to, retinoids, antibiotics, benzoyl peroxide, Vitamin E, salicylic acid, hydroxy acids, fatty acids or other agents can be introduced. If desired, the acne region can first be cleansed, stretched or subjected to other treatments prior to pressure isolation. In addition, pressure isolation treatment of acne may be administered along with phototherapy, medications, topical agents or other treatment methods and protocols.

In some embodiments, the devices and methods described herein can be used to treat discoid lupus erythematosus (DLE). DLE is characterized by attack of healthy cells by the immune system and can result in permanent tissue scarring. The devices and methods described herein can be used to pressure isolate DLE regions and treat them to reduce the level of scarring. For example, one or more DLE regions can be pressure isolated. The pressure isolated regions can receive a therapeutic agent (in a treatment volume) for a treatment period. The treatment volume can then be removed, and the pressure can be released. Illustrative agents that can be introduced into pressure isolated DLE regions include but are not limited to, corticosteroids, antimalarials, calcineurin inhibitors, retinoids, imiquimod, methotrexate, mycophenolate, mofetil, thalidomide or monoclonal antibodies such as etanercept can be administered. In instances where a biological agent is introduced, it may not be desirable to remove the biological agent post-injection. Instead, the biological agent may be permitted to circulate systemically, if desired. In some instances, the devices and methods described herein may be co-administered with other treatment methods and protocols to treat DLE regions.

In certain instances, the devices and methods described herein can be used to treat cold sores. Cold sores are typically caused by the herpes simplex virus and exhibit small sores or blisters around the mouth. The devices and methods described herein can be used to pressure isolate cold sores. For example, one or more cold sores can be pressure isolated. The pressure isolated region can receive a therapeutic agent (in a treatment volume) for a treatment period. The treatment volume can then be removed, and the pressure can be released. Illustrative agents that can be introduced into pressure isolated cold sores include, but are not limited to, docosonal, acyclovir, penciclovir, famciclovir, valaciclovir, lysine or other antiviral, amino acid or chemical agents can be used. In instances where a cold sore is treated, it may not be desirable to remove the agent post-injection. Instead, the agent may be permitted to circulate systemically, if desired. In some instances, the devices and methods described herein may be co-administered with other treatment methods and protocols to treat cold sores.

In certain embodiments, the devices and methods described herein can be used to treat rosacea. Rosacea is characterized by redness and may be accompanies by pimples. The redness often occurs on the face, but it may also occur on the neck, chest, ears and scalp. The devices and methods described herein can be used to pressure isolate rosacea regions. For example, one or more rosacea regions can be pressure isolated. The pressure isolated region can receive a therapeutic agent (in a treatment volume) for a treatment period. The treatment volume can then be removed, and the pressure can be released. Illustrative agents that can be introduced into pressure isolated rosacea regions include, but are not limited to, anti-angiogenesis agents, antibiotics, hydroxy acids, retinoids, vitamin E, clonidine, nadolol, propranolol, antihistamines, methylsulfonylmethane, silymarin and other agents. Pressure isolation treatment may also be performed in combination with phototherapy or other treatment methods and protocols.

In certain embodiments, the devices and methods described herein can be used to treat eczema or dermatitis. Eczema is characterized by itchy skin that becomes crusty and/or red. The devices and methods described herein can be used to pressure isolate eczema regions. For example, one or more eczema regions can be pressure isolated. The pressure isolated region can receive a therapeutic agent (in a treatment volume) for a treatment period. The treatment volume can then be removed, and the pressure can be released. Illustrative agents that can be introduced into pressure isolated eczema regions include, but are not limited to, corticosteroids, immunosuppressants, retinoids or other agents. Pressure isolation treatment may also be performed in combination with phototherapy or other treatment methods and protocols.

In certain embodiments, the devices and methods described herein can be used to treat vitiligo. Vitiligo is characterized by depigmentation of parts of the skin and may be caused by autoimmune responses. The devices and methods described herein can be used to pressure isolate vitiligo regions. For example, one or more vitiligo regions can be pressure isolated. The pressure isolated region can receive a therapeutic agent (in a treatment volume) for a treatment period. The treatment volume can then be removed, and the pressure can be released. Illustrative agents that can be introduced into pressure isolated vitiligo regions include, but are not limited to, immunomediators, immunomodulators, corticosteroids, immunosuppressants, pigments, or other agents. Pressure isolation treatment may also be performed in combination with pigment replacement, bleaching, phototherapy, melanocyte transplantation or other treatment methods and protocols.

In certain embodiments, the devices and methods described herein can be used to treat necrotizing fasciitis. Necrotizing fasciitis is characterized by infection of deeper layer of the skin and subcutaneous tissues. The devices and methods described herein can be used to pressure isolate necrotizing fasciitis regions. For example, one or more necrotizing fasciitis regions can be pressure isolated. The pressure isolated region can receive a therapeutic agent (in a treatment volume) for a treatment period. The treatment volume can then be removed, and the pressure can be released. Illustrative agents that can be introduced into pressure isolated necrotizing fasciitis regions include, but are not limited to, antibiotics, antifungals, antivirals and other agents. Pressure isolation treatment may also be performed in combination with surgical debridement, hyperbaric oxygen or other treatment methods and protocols.

In certain examples, the devices and methods described herein can be used to treat candidiasis. Candidiasis is characterized by infection of tissue by yeast with accompanying inflammation and discomfort. The devices and methods described herein can be used to pressure isolate candidiasis regions. For example, one or more candidiasis regions can be pressure isolated. The pressure isolated region can receive a therapeutic agent (in a treatment volume) for a treatment period. The treatment volume can then be removed, and the pressure can be released. Illustrative agents that can be introduced into pressure isolated candidiasis regions include, but are not limited to, antifungals, fatty acids, dyes or other agents. Pressure isolation treatment may also be performed in combination with topical treatments or other treatment methods and protocols.

In certain embodiments, the devices and methods described herein can be used to treat carbuncle. Carbuncle is characterized by a large abscess that drains pus. Carbuncle is typically caused by a bacterial infection. The devices and methods described herein can be used to pressure isolate carbuncle regions. For example, one or more carbuncles can be pressure isolated. The pressure isolated region can receive a therapeutic agent in a treatment volume for a treatment period. The treatment volume can then be removed, and the pressure can be released. Illustrative agents that can be introduced into pressure isolated carbuncle regions include, but are not limited to, antibiotics or other agents. Pressure isolation treatment may also be performed to drain the carbuncle prior to and/or after introduction of a therapeutic agent.

In certain instances, the devices and methods described herein can be used to treat cellulitis. Cellulitis is characterized by a bacterial infection of the dermis and subcutaneous fat. The devices and methods described herein can be used to pressure isolate cellulitis regions. For example, one or more cellulitis regions can be pressure isolated. The pressure isolated region can receive a therapeutic agent in a treatment volume for a treatment period. The treatment volume can then be removed, and the pressure can be released. Illustrative agents that can be introduced into pressure isolated cellulitis regions include, but are not limited to, antibiotics, NSAIDs, aspirin or other agents. Pressure isolation treatment may also be performed in combination with debridement or surgical removal of the dead tissue.

In certain embodiments, the devices and methods described herein can be used to treat hyperhidrosis. Hyperhidrosis is characterized by excessive sweating. The devices and methods described herein can be used to pressure isolate hyperhidrosis regions. For example, one or more hyperhidrosis regions can be pressure isolated. The pressure isolated region can receive a therapeutic agent in a treatment volume for a treatment period. The treatment volume can then be removed, and the pressure can be released. Illustrative agents that can be introduced into pressure isolated hyperhidrosis regions include, but are not limited to, a botulinum toxin or similar bacterial toxins, salts such as aluminum chloride, anticholinergic drugs (e.g., oxybutynin, glycopyrrolate, propantheline bromide, benztropine, etc.) or other agents can be used.

In certain examples the devices and methods described herein can be used to treat impetigo. Impetigo is characterized by infection of the skin by bacteria. The devices and methods described herein can be used to pressure isolate impetigo regions. For example, one or more impetigo regions can be pressure isolated. The pressure isolated region can receive a therapeutic agent in a treatment volume for a treatment period. The treatment volume can then be removed, and the pressure can be released. Illustrative agents that can be introduced into pressure isolated impetigo regions include, but are not limited to, antibiotics or other agents.

In certain embodiments, the devices and methods described herein can be used to treat decubitis ulcer or pressure ulcers or bedsores. Decubitis ulcer is characterized by injury to the skin and/or underling tissue as a result of pressure. The devices and methods described herein can be used to pressure isolate decubitis ulcer regions. For example, one or more decubitis ulcers can be pressure isolated. The pressure isolated region can receive a therapeutic agent in a treatment volume for a treatment period. The treatment volume can then be removed, and the pressure can be released. Illustrative agents that can be introduced into pressure isolated decubitis ulcer regions include, but are not limited to, antibiotics, NSAIDs, muscle relaxants or other agents. The application of negative pressure in combination with pressure isolated introduction of a therapeutic may further assist in treatment of decubitis ulcers.

In certain examples, the devices and methods described herein can be used to treat erysipelas. Erysipelas is characterized by acute infection of the upper dermis usually caused by bacteria. The devices and methods described herein can be used to pressure isolate erysipelas regions. For example, one or more erysipelas regions can be pressure isolated. The pressure isolated region can receive a therapeutic agent in a treatment volume for a treatment period. The treatment volume can then be removed, and the pressure can be released. Illustrative agents that can be introduced into pressure isolated erysipelas regions include, but are not limited to, antibiotics, NSAIDs or other agents.

In certain embodiments, the devices and methods described herein can be used to treat dermatomyositis. Dermatomyositis is characterized by inflammation of the muscles and skin. The devices and methods described herein can be used to pressure isolate dermatomyositis regions. For example, one or more dermatomyositis regions can be pressure isolated. The pressure isolated region can receive a therapeutic agent in a treatment volume for a treatment period. The treatment volume can then be removed, and the pressure can be released. Illustrative agents that can be introduced into pressure isolated dermatomyositis regions include, but are not limited to, corticosteroids, immunosuppressants, methotrexate, mycophenolate, immunoglobins, azathioprine, cyclophosphamide, rituximab or other chemical or biological agents. Where a biological agent is used, it may be desirable to leave the biological agent after pressure isolation is discontinued.

In certain examples, the devices and methods described herein can be used to treat molluscum contagiosum. Molluscum contagiosum is characterized by viral infection of the skin or mucous membranes. The devices and methods described herein can be used to pressure isolate molluscum contagiosum regions. For example, one or more molluscum contagiosum lesions can be pressure isolated. The pressure isolated lesion can receive a therapeutic agent in a treatment volume for a treatment period. The treatment volume can then be removed, and the pressure can be released. Illustrative agents that can be introduced into pressure isolated molluscum contagiosum lesions include, but are not limited to, antivirals, cantharidin, salicylic acid, imiquimod, retinoids, plant oils or other agents.

In certain embodiments, the devices and methods described herein can be used to treat cysts, e.g., sebaceous cyst, epidermoid cysts, pilar cysts, etc. A cyst is characterized by a mass of tissue. The devices and methods described herein can be used to pressure isolate cysts. For example, one or more cysts can be pressure isolated. The pressure isolated region can receive a therapeutic agent in a treatment volume for a treatment period. The treatment volume can then be removed, and the pressure can be released. Illustrative agents that can be introduced into pressure isolated cysts include, but are not limited to, hydrogen peroxide, antibiotics, anesthetics, and other agents. Pressure isolation can be performed in combination with surgical excision, if desired.

In certain examples, the devices and methods described herein can be used to treat seborrheic keratosis. Seborrheic keratosis is a non-cancerous growth that begins in keratinocytes. The devices and methods described herein can be used to pressure isolate seborrheic keratosis regions. For example, one or more seborrheic keratosis regions can be pressure isolated. The pressure isolated region can receive a therapeutic agent in a treatment volume for a treatment period. The treatment volume can then be removed, and the pressure can be released. Illustrative agents that can be introduced into pressure isolated seborrheic keratosis regions include, but are not limited to, liquid nitrogen, carbon dioxide, argon or other agents.

In certain embodiments, the devices and methods described herein can be used to treat keloid. Keloid is characterized by scar formation composed mainly of collagen. The devices and methods described herein can be used to pressure isolate keloid regions. For example, one or more keloid regions can be pressure isolated. The pressure isolated region can receive a therapeutic agent in a treatment volume for a treatment period. The treatment volume can then be removed, and the pressure can be released. Illustrative agents that can be introduced into pressure isolated keloid regions include, but are not limited to, corticosteroids, collagenase or other agents.

In certain instances, the devices and methods described herein can be used to treat lichen planus. Lichen planus is characterized by redness and scaling of the skin as a result of an autoimmune response. The devices and methods described herein can be used to pressure isolate lichen planus regions. For example, one or more lichen planus regions can be pressure isolated. The pressure isolated region can receive a therapeutic agent in a treatment volume for a treatment period. The treatment volume can then be removed, and the pressure can be released. Illustrative agents that can be introduced into pressure isolated lichen planus regions include, but are not limited to, corticosteroids, vitamin D3 analogs, antibiotics, anti-coagulants, retinoids, or other agents. Pressure isolation treatment may also be performed in combination with phototherapy and/or cryotherapy.

In certain embodiments, the devices and methods described herein can be used to treat actinic keratosis. Actinic keratosis is a premalignant condition of thick, scaly skin patches. The devices and methods described herein can be used to pressure isolate actinic keratosis regions. For example, one or more actinic keratosis regions can be pressure isolated. The pressure isolated region can receive a therapeutic agent in a treatment volume for a treatment period. The treatment volume can then be removed, and the pressure can be released. Illustrative agents that can be introduced into pressure isolated actinic keratosis regions include, but are not limited to, 5-fluorouracil, NSAIDs, imiquimod or other agents. Pressure isolation treatment may also be performed in combination with phototherapy and/or cryotherapy.

In certain instances, the devices and methods described herein can be used to treat skin rashes from shingles. For example, the herpes zoster virus can cause painful blisters of the skin typically in a striped fashion. The devices and methods described herein can be used to pressure isolate various areas of the skin rash from shingles. The pressure isolated region can receive a therapeutic agent in a treatment volume for a treatment period. The treatment volume can then be removed, and the pressure can be released. Illustrative agents that can be introduced into pressure isolated shingles skin rash regions include, but are not limited to, NSAIDs, antivirals (acyclovir, valaciclovir, famciclovir), corticosteroids, interferon, immunoglobins, antibodies or other agents. A linear array of pressure members may be particularly desirable for use in treating shingles skin rashes based on the linear nature of the rash.

In certain embodiments, the devices and methods described herein can be used to treat roundworm, skin ulcers, corns, calluses, tinea versicolor, pemphigoid, shingles or other skin disorders. The devices and methods described herein can be used to pressure isolate various areas of the skin comprising these disorders. The pressure isolated region can receive a therapeutic agent in a treatment volume for a treatment period. The treatment volume can then be removed, and the pressure can be released. Illustrative agents that can be introduced into pressure isolated skin regions include, but are not limited to, antibiotics, antifungals, antivirals, corticosteroids, immunosuppressants, interferon, immunoglobins, antibodies or other agents.

In certain configurations, the devices and methods described herein can also be used to pressure isolate and introduce agents at the site of tick bites, mosquito bites, spider bites or snake bites if desired. For example, systemically toxic amounts of an anti-parasitic drug can be introduced into a pressure isolated skin region immediately after removal of a tick to reduce the likelihood of tick borne infections, e.g., Lyme disease or Rocky Mountain spotted fever.

Systemically toxic amounts of an anti-malarial drug, an antiviral, interferon or other drug can be introduced into a pressure isolated skin region immediately after a mosquito bite to reduce the likelihood of mosquito borne infections, e.g., malaria, West Nile Virus, EEE virus, dengue virus, etc. Similarly, systemically toxic amounts of anti-venom can be introduced at the site of a pressure isolated snake bite to counteract the snake venom.

While the exact therapeutic agent introduced into the isolated lesion or region described herein can vary, as noted herein the therapeutic agent is typically introduced in a treatment volume for a treatment period. After the treatment period, a volume about the same as the treatment volume can then be removed, e.g., about 90% or more of the treatment volume is removed. Alternatively, the agent may be perfused through the region at a systemically toxic concentration (or higher) during the treatment period or some portion thereof. Illustrative therapeutic agents that can be introduced into pressure isolated regions include, but are not limited to, anti-cancer and/or anti-neoplastic agents such as, for example, bleomycin, methotrexate or aminopterin. Illustrative other therapeutic agents include, but are not limited to, a neurotransmitter, a cholinase activator or inhibitor, an acetylcholinesterase activator or inhibitor, an acetylcholine, an acetylcholine agonist or derivative, a nicotinic receptor agonist, a nicotinic receptor antagonist, a muscarinic receptor agonist, a muscarinic receptor antagonist, dopamine, a dopamine derivative, a catecholamine, an adrenergic receptor agonist, an adrenergic receptor antagonist, an anticholinesterase agent, a nicotinic cholinergic receptor agonist, a nicotinic cholinergic receptor antagonist, a ganglionic blocking compound, a ganglionic stimulant, a serotonin receptor agonist, a serotonin receptor antagonist, an ion channel agonist, an ion channel antagonist, a neuromodulator, a therapeutic gas (e.g., oxygen, carbon dioxide, nitric oxide), ethanol or an ethanol derivative, a benzodiazepine analog, a phenothiazine, a thioxanthene, a heterocyclic compound, a sedative, a norepinephrine reuptake inhibitor, an antidepressant, a serotonin reuptake inhibitor, a monoamine oxidase agonist, a monoamine oxidase antagonist, a sodium ion channel activator, a sodium ion channel inhibitor, a calcium ion channel activator, a calcium ion channel inhibitor, a hydantoin, a barbituate, a stilbene, an iminostilbene, a succinimide, an oxazolidinedione, an antiseizure agent, an analgesic, an opioid, a peptide, an opioid agonist, an opioid antagonist, an autocoid, histamine, a histamine analog, a H1-receptor agonist, a H1-receptor antagonist, a H3-receptor agonist, a H3-receptor antagonist, an eicosanoid, a prostaglandin, a leukotriene, an antiinflammatory agent, an antipyretic, a nonsteroidal antiinflammatory agent, a salicylic acid derivative, a salicylate, a para-aminophenol derivative, an indole, an indene, an indole acetic acid, an indene acetic acid, a heteroaryl acetic acid, a propionic acid, an arylpropionic acid, an anthranilic acid, an enolic acid, an alkanone, an oxicam, gold and gold derivatives, a uricosuric agent, a corticosteroid, a bronchodilator, a diuretic, a vasopressin receptor agonist, a vasopressin receptor antagonist, angiotensin, an angiotensin analog, renin, a renin analog, an inhibitor of the renin-angiotensin system, an angiotensin receptor agonist, an angiotensin receptor antagonist, a renin inhibitor, an endopeptidase inhibitor, an organic nitrate, a calcium channel blocker, a beta-adrenergic receptor antagonist, an alpha-adrenergic receptor antagonist, an antiplatelet agent, an antithrombotic agent, an antihypertensive, a benzothiadiazine, a sympatholytic agent, a vasodilator, an angiotensin converting enzyme inhibitor, an angiotensin II receptor antagonist, a cardiac glycoside, a dopaminergic receptor agonist, a phosphodiesterase inhibitor, an antiarrhythmic drug, an HMG CoA reductase inhibitor, an H2 histamine receptor antagonist, an antibiotic, a hydrogen-potassium ATPase inhibitor, an antacid, a laxative, an antidiarrheal agent, an antiemetic agent, a prokinetic agent, oxytocin, an antimalarial agent, a diaminopyrimidine, quinine and quinine derivatives, quinoline and quinoline derivatives, an antihelminthic agent, an antimicrobial agent, a sulfonamide, a quinolone, a penicillin, a cephalosporin, a beta-lactam, an aminoglycoside, a tetracycline, a chloramphenicol, an erythromycin, an isonicotinic acid compound and derivatives thereof, a macrolide, a sulfone, an antifungal agent, an imidazole, a triazole, an antiviral agent, a protease inhibitor, an antiretroviral agent, a reverse transcriptase inhibitor, an acyclic nucleoside phosphonate, a nitrogen mustard, an ethylenimine, a methylmelamine, an alkyl sulfonate, a nitrosourea, a triazene, a folic acid analog, a pyrimidine analog, a purine analog, a vinca alkaloid, an epipodophyllotoxin, a coordination complex, a platinum coordination complex, an anthracenedione, a substituted urea, a methylhydrazine derivative, an adrenocortical suppressant, a progestin, an estrogen, an antiestrogen, an androgen, an antiandrogen, a gonadotropin-releasing hormone analog, an immunosuppressant, an interferon, a granulocyte macrophage-colony stimulating factor, a tumor necrosis factor, an interleukin, an antibody, an antigen, a hematopoietic agent, an anticoagulant, a hormone, a growth hormone, a glucocorticoid, an antiseptic, insulin, a hypoglycemic agent, a hyperglycemic agent, an insulin analog, a vitamin, a water soluble vitamin, a fat soluble vitamin, a skin agent, an ocular agent, a cosmetic agent, a heavy metal antagonist, or other suitable synthetic or non-synthetic therapeutics. Where a therapeutic is present, it may be present alone or in combination with a biological agent or another therapeutic.

In some instances, a biological agent such as an enzyme, antibody, antigen, growth factor, protein factors, clotting factors or other biological agents can be introduced into a pressure isolated tissue, e.g., skin lesion or skin region. For example, a monoclonal antibody, an anti-sense nucleic acid, an interfering RNA (RNAi), a virus or other biological agents can be introduced into the isolated lesion or region.

In certain configurations, after the material is introduced into the isolated lesion or region, it may remain for a selected treatment period, e.g., 30 seconds, 1 minute, 2 minutes, 5 minutes, 10 minutes, 1 hour or more, and then the agent can be removed. For example, where the material is introduced in a systemically toxic concentration (or higher) into a pressure isolated region, pressure isolation slows or prevents perfusion out of the pressure isolated lesion or region. The introduced material may remain resident for an effective period (or circulated into and out of the pressure isolated lesion) to treat the lesion or condition and can then be removed by suction, external pressure or other means. In some instances, at least 50% of the material introduced into the isolated lesion or region is subsequently removed after the treatment period, more particularly, at least 60%, 65%, 70%, 75%, 80%, 85%, 90%, 95% or more of the material can be removed after treatment.

While the devices described herein can be used to isolate skin lesion or regions, the pressure applied can be periodically released to permit perfusion into the lesion or area such that oxygen or nutrients are not completely shielded from entry into the skin lesion or region.

In some instances, the devices and methods can also be used to introduce cosmetic agents into isolated skin areas to reduce wrinkles, age spots, skin discoloration or other cosmetic issues commonly associated with the skin.

In certain embodiments, the various components described herein can be packaged in a kit. For example, a kit may comprise a pressure member and instructions for using the pressure member to pressure isolate a tissue disorder. The kit may also include one or more of a needle, syringe, a therapeutic agent, a second pressure member or other components.

Certain specific examples are described in more detail below to illustrate further some of the aspects described herein.

### Example 1

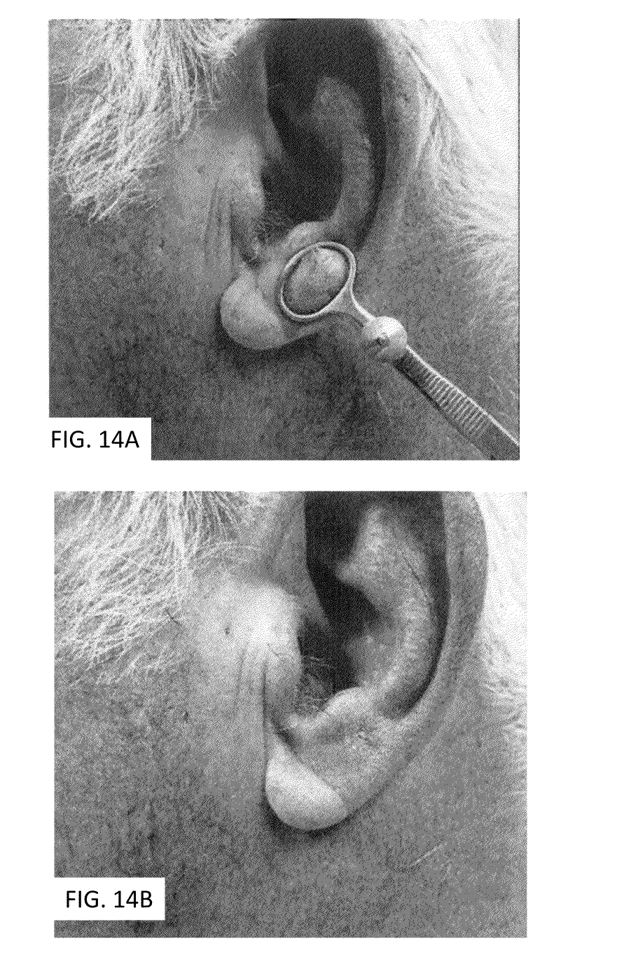

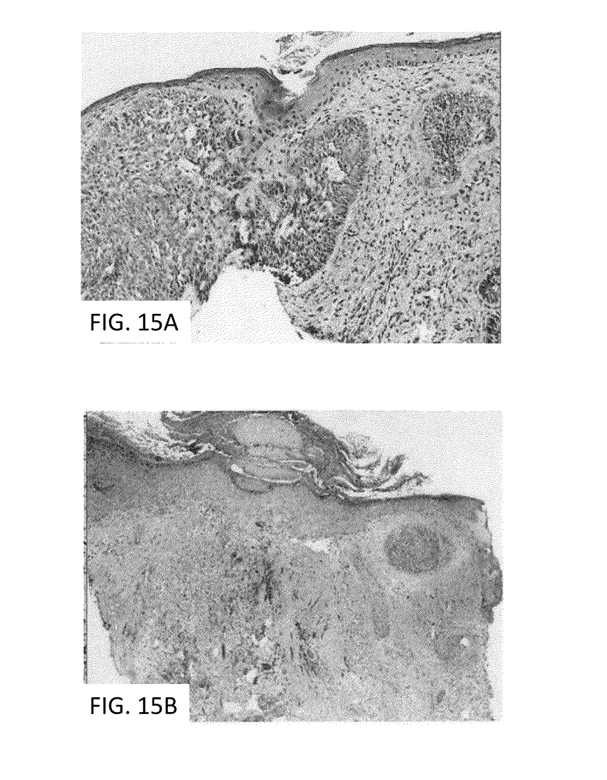

A 69-year old with basal cell carcinoma (one year history of asymptomatic growth) on his left ear lobe was treated by mechanically isolating the basal cell carcinoma (BCC). Diagnosis of BCC was made based on the lesion's clinical presentation as a well-circumscribed pearly nodule with central crusted ulceration (see FIG. 14A) and the results of a shave biopsy (see FIG. 15A). The patient refused surgical therapy.

A chalazion clamp was used to isolate the tissue surrounding the BCC. One fifth unit (0.2 cc×1 unit/cc) of bleomycin in 1% lidocaine was injected into the clamped region (systemic bleomycin concentrations are typically in the range of 0.00023 units/mL or about 1000×lower). The injected bleomycin remained within the clamped area as evidenced by a localized raised and stretched area of the skin (see FIG. 14A). After 20 minutes, an 18-gauge needle was used to remove most of the injected fluid from the clamped area. The chalazion clamp was then removed. The patient only complained of minor discomfort (no adverse effects). At 25-days post-treatment, the BCC appeared clinically cured (see FIG. 14B). The clinical result was confirm by histology from a 4 mm punch biopsy (see FIG. 15B).

### Example 2

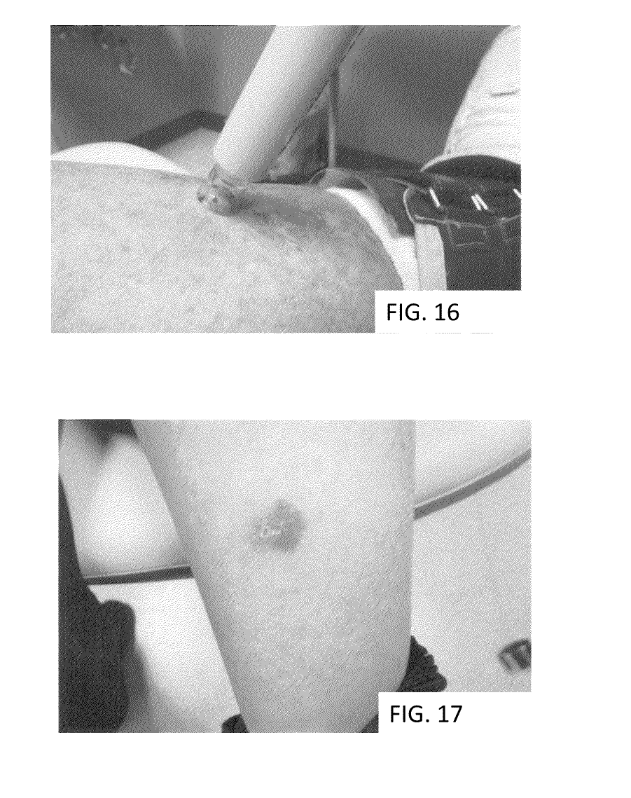

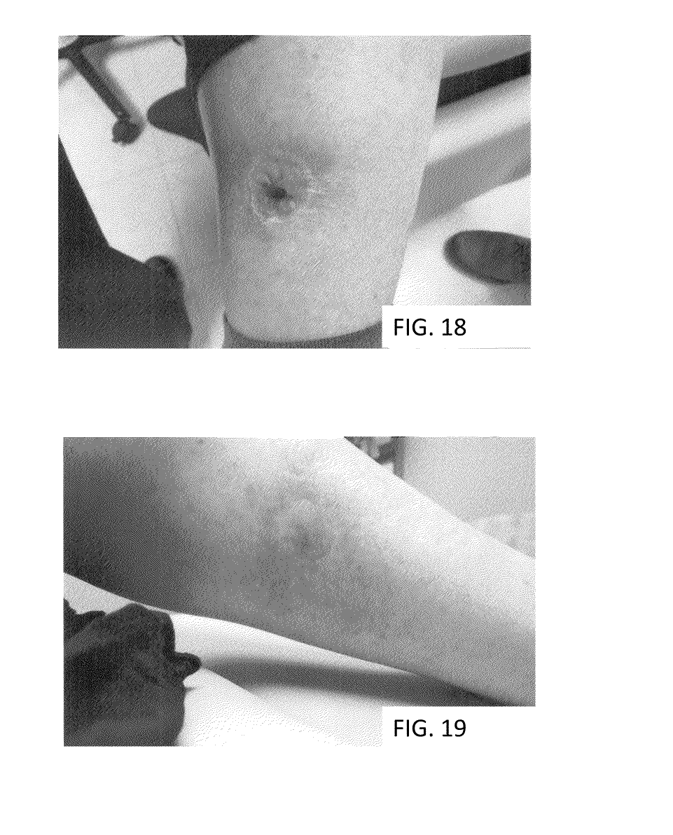

FIGS. 16-19 are photographs showing the course of treatment of a superficial basal cell carcinoma (BCC) using similar methods described in Example 1. FIG. 16 is a photograph showing the area during the treatment with the pressure isolation device (which applied suction) placed over the area. FIG. 17 is a photograph showing the area immediately after treatment. FIG. 18 shows the area 24 hours after treatment, and FIG. 19 shows the area 3 weeks after treatment. The BCC appeared clinically cured following the treatment.

When introducing elements of the examples disclosed herein, the articles “a,” “an,” “the” and “said” are intended to mean that there are one or more of the elements. The terms “comprising,” “including” and “having” are intended to be open-ended and mean that there may be additional elements other than the listed elements. Although certain aspects, examples and embodiments have been described above, it will be recognized by the person of ordinary skill in the art, given the benefit of this disclosure, that additions, substitutions, modifications, and alterations of the disclosed illustrative aspects, examples and embodiments are possible.

1. A method of treating a skin lesion, the method comprising:
increasing local pressure within a skin lesion by pressure isolation of the skin lesion;
intralesionally introducing a therapeutic agent into the pressure isolated skin lesion, in which the therapeutic agent is introduced in at least an effective amount to treat the skin lesion.

2. The method of claim 1, further comprising clamping the skin lesion to pressure isolate the skin lesion.

3. The method of claim 1, further comprising providing hydraulic pressure to the skin lesion to pressure isolate the skin lesion.

4. The method of claim 3, further comprising configuring the hydraulic pressure to be a positive hydraulic pressure.

5. The method of claim 1, further comprising providing pneumatic pressure to the skin lesion to pressure isolate the skin lesion.

6. The method of claim 5, further comprising configuring the pneumatic pressure to be a negative pneumatic pressure.

7. The method of claim 1, in which the introducing step comprising introducing the therapeutic agent in a treatment volume, and in which the method further comprises removing about a same volume as the treatment volume from the skin lesion after a first treatment period.

8. The method of claim 7, further comprising reintroducing the intralesionally introduced therapeutic agent for a second treatment period after the first treatment period.

9. The method of claim 1, further comprising selecting the therapeutic agent to be an agent that is systemically toxic.

10. The method of claim 1, further comprising introducing the therapeutic agent into the pressure isolated skin lesion in a concentration that is at least 10 times greater than a systemically toxic concentration used for the therapeutic agent.

11. The method of claim 1, further comprising intralesionally introducing a second therapeutic agent different from the first therapeutic agent.

12. The method of claim 1, in which the skin lesion comprises basal cell carcinoma and the therapeutic agent is bleomycin.

13. The method of claim 12, in which the bleomycin is introduced into the skin lesion at a concentration at least 10 times greater than a systemically toxic concentration of bleomycin.

14. The method of claim 1, in which the pressure isolation step comprises clamping the skin lesion between a first mechanical pressure member and a second mechanical pressure member.

15. The method of claim 1, in which the pressure isolation step comprises providing a positive pressure by engaging the skin lesion with a first mechanical pressure member.

16. The method of claim 1, in which the pressure isolation step comprises providing a negative pressure using a negative pneumatic pressure.

17. The method of claim 16, in which the negative pneumatic pressure is provided by drawing the skin lesion into a vacuum device to pressure isolate the skin lesion.

18. The method of claim 1, in which the pressure isolation step comprises providing a positive pressure by engaging the skin lesion with a first pressure member, in which the first pressure member is configured to expand upon application of a hydraulic force to pressure isolate the skin lesion.

19. The method of claim 1, in which the pressure isolation step comprises providing a negative pressure by engaging the skin lesion with a first pressure member, in which the first pressure member is configured to contract upon application of a negative hydraulic force to pressure isolate the skin lesion.

20. The method of claim 1, in which the skin lesion comprises basal cell carcinoma and the therapeutic agent is bleomycin, in which the bleomycin is introduced into the skin lesion at a concentration at least 10 times greater than a systemically toxic concentration of bleomycin, and in which the introduced bleomycin is substantially removed from the skin lesion after a first treatment period.

21-79. (canceled)
